(19) United States
(12) Patent Application Publication
(10) Pub. No.: US 2026/0144712 A1
(43) Pub. Date: May 28, 2026

(54) NASOGASTRIC TUBE, AND ASSOCIATED SYSTEMS AND METHODS

(71) Applicant: Julie Seavello, Mill Creek, WA (US)

(72) Inventor: Julie Seavello, Mill Creek, WA (US)

(21) Appl. No.: 19/114,701

(22) PCT Filed: Nov. 20, 2023
(86) PCT No.: PCT/US2023/080462

§ 371 Date: Mar. 24, 2025

Related U.S. Application Data
(60) Provisional application No. 63/384,717, filed on Nov. 22, 2022.

(51) Int. Cl. A61J 7/00; A61M 1/00; A61M 16/06

(52) U.S. Cl. CPC A61J 7/0038; A61M 1/742; A61M 1/85; A61M 1/87; A61M 16/0672; A61M 16/0683; A61M 2202/048; A61M 2205/583; A61M 2210/0618; A61M 2210/065; A61M 2210/1053

(57) ABSTRACT

Nasogastric tubes, and associated systems and methods are described. In one embodiment, a device for administering therapeutic agent to a patient's target area includes: a nasal anchor configured for insertion into a nose of the patient. The nasal anchor includes a first pocket in fluid communication with at least one first opening configured for delivering a first therapeutic agent to a first target area of the patient. The device also includes a nasogastric tube configured for insertion through the nasal anchor into a stomach of the patient; and a wrapping disposed on the nasogastric tube. The wrapping includes a second pocket in fluid communication with at least one second opening configured for delivering a second therapeutic agent to a second target area of the patient.

### CROSS-REFERENCE TO RELATED APPLICATION

This application is a national phase of International Application No. PCT/US2023/080462, filed Nov. 20, 2023, which claims the benefit of U.S. Provisional Application No. 63/384,717, filed Nov. 22, 2022, the entire disclosures of which are hereby incorporated by reference in their entirety.

### BACKGROUND

Nasogastric tubes can serve a wide range of functions. For instance, nasogastric tubes are used for administering fluids or gases, or assisting in draining of bodily fluids, for example, stomach fluid.

Nasogastric tubes can be inserted through patient's nose into patient's stomach to evacuate contents of patient's stomach. Patient's stomach may be evacuated for different reasons, for example, to remove excess accumulation of stomach fluid, as a part of poisoning treatment, to remove blood associated with stomach ulcers, etc. However, nasogastric tube insertion may cause tissue irritation, scratching or bleeding along the insertion path. As a result, at a minimum the patient experiences discomfort, and may possibly also experience pain and difficult breathing. Such side effects in turn affect the very medical procedure that relies on the nasogastric tube.

With some conventional technologies, physicians attempt to apply topical anesthetics to the affected tissue. However, this approach is typically only useful if the location of affected area is both known and accessible before the nasogastric tube insertion, which may not always be the case. With some other conventional technologies, local anesthetic may be applied to the outside surface of nasogastric tube prior to the insertion. However, with this technology it is difficult to control where exactly is the anesthetic applied. For example, during the insertion some anesthetic may be removed by rubbing against patient's tissue that in turn receives significant amount of anesthetic, which, as a result, is not available to be applied at its target area.

Accordingly, improved nasogastric tubes and associated insertion methods are needed.

### SUMMARY

This summary is provided to introduce a selection of concepts in a simplified form that are further described below in the Detailed Description. This summary is not intended to identify key features of the claimed subject matter.

Briefly, the inventive technology is directed to a controlled application of local anesthetics or other therapeutic agent at predetermined locations of patient's airway. In some embodiments, one such area is patient's nasal area and the other area is an oropharyngeal region of patient's mouth, as these areas may be irritated and/or scratched during the insertion of the nasogastric tube.

In some embodiments, the inventive device includes a combination of a nasal anchor and a nasogastric tube. The nasal anchor may be first inserted into the patient's airway to guide a subsequent insertion of the nasogastric tube, thus preventing or at least reducing tissue irritation along the length of the nasal anchor. The nasal anchor may include longitudinally disposed space (also referred to as a pocket) for holding and releasing of the therapeutic agents (e.g., anesthetic, medication, etc.). In operation, the therapeutic agents are transferred into the pockets, and then released toward the target tissue through the openings in the pockets. Such openings may be micro-apertures that face the target tissue when the nasal anchor and the nasogastric tube are in their operational position. Increasing the pressure inside the pockets for therapeutic agents pushes the therapeutic agent through the micro-apertures toward the target tissue. In some embodiments, the therapeutic agent may be delivered using detachable medication straws.

In some embodiments, the therapeutic agents may also be applied to another area of the patient's tissue, for example to the oropharynx area, through a wrapping having a space (a pocket) with openings that touch the patient's tissue. The therapeutic agents may be delivered to the pocket through a lumen that terminates in a Luer lock for easier control of the therapeutic agent delivery.

In some embodiments, the nasogastric tube includes a visual marker (e.g., a radio-opaque line) extending along an anterior side of the nasogastric tube. The operator may use the marker line to properly align the nasogastric tube against the nasal anchor or against patient's anatomy, therefore assuring proper orientation of the therapeutic agent release. The nasogastric tube may include a strengthening element extending along its posterior side to increase rigidity of the nasal anchor, therefore enabling easier and more controllable rotational positioning of the nasal anchor and the nasogastric tube with respect to the target tissue of the patient. In some embodiments, the strengthening element is made of high strength polyurethane or other material that is more rigid than the surrounding material.

In some embodiments, the nasogastric tube includes a shaped inflatable balloon at its distal end. In operation, the inflatable balloon lifts the openings for the suction and sump venting functions away from the gastric mucosa.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIG. 1 is a cross-section of a nasogastric tube in accordance with prior art;

FIG. 2 is a side view of a nasogastric tube assembly in accordance with an embodiment of the present technology;

FIG. 3 is a cross-sectional view A-A of a combination of nasogastric tube and nasal anchor shown in FIG. 2;

FIG. 3A is a side view of a combination of nasal anchor and nasogastric tube in accordance with an embodiment of the present technology;

FIG. 3B is a cross-sectional side view of a combination of nasal anchor and nasogastric tube in accordance with embodiments of the present technology;

FIG. 3C is a cross-sectional side view of therapeutic agent delivery in accordance with embodiments of the present technology;

FIG. 4 is a partial side view of a nasogastric tube in accordance with an embodiment of the present technology;

FIGS. 5A and 5B are detail views of Luer locks in accordance with embodiments of the present technology;

FIG. 6 is a cross-sectional view B-B of a combination of nasogastric tube and nasal anchor shown in FIG. 2;

FIG. 7 is a cross-sectional view C-C of a combination of nasogastric tube and nasal anchor shown in FIG. 2;

FIG. 8 is a partial side view of nasogastric tube in accordance with an embodiment of the present technology;

FIG. 9 is an isometric view of an attachment of a nasal anchor and nasogastric tube in accordance with an embodiment of the present technology;

FIG. 9A is a detail view of an attachment of a nasal anchor in accordance with an embodiment of the present technology;

FIG. 10 is a detail view of an attachment of a nasal anchor in accordance with an embodiment of the present technology; and

FIG. 11 is a flowchart of a method for inserting and using a nasogastric tube in accordance with an embodiment of the present technology.

### DETAILED DESCRIPTION

While several embodiments have been illustrated and described, it will be appreciated that various changes can be made therein without departing from the spirit and scope of the claimed subject matter.

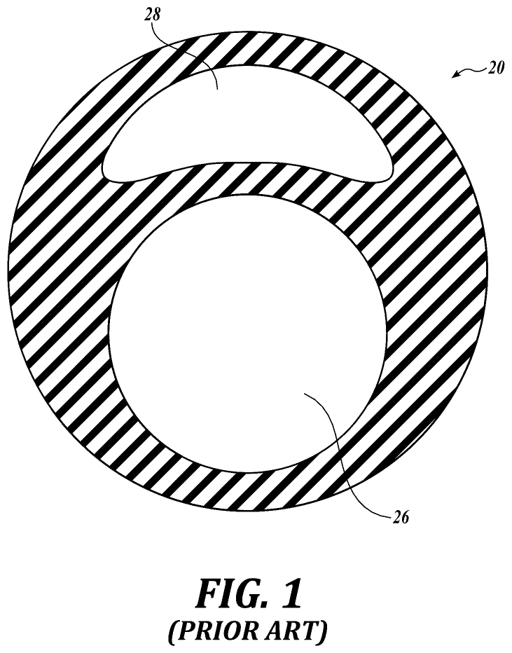

FIG. 1 is a cross-section of a nasogastric tube in accordance with prior art. Nasogastric tube 20 includes lumen 26 that is used for evacuating stomach fluid. Lumen 28 is a gas lumen for providing gas to patient's stomach to counteract negative pressure inside patient's stomach that is created by evacuation of the stomach fluid. Lumen 26 is typically a larger one of the two illustrated lumens. However, the insertion process and the presence of the nasogastric tube 20 inside the patient's airwave may cause irritation and pain along the path of the nasogastric tube.

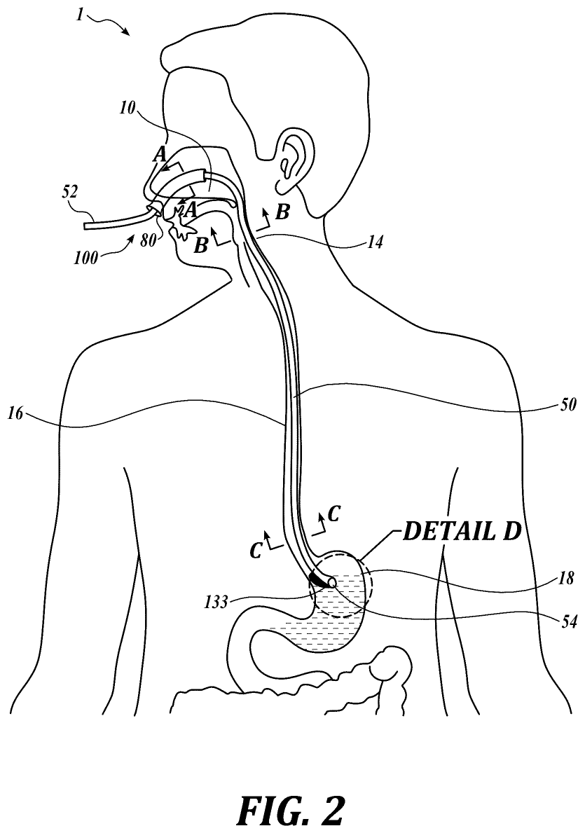

FIG. 2 is a side view of a nasogastric tube assembly in accordance with an embodiment of the present technology. In some embodiments of the inventive technology, the nasogastric tube assembly 100 includes a nasal anchor 80 that is combined with the inventive nasogastric tube 50 to alleviate negative effects of the conventional nasogastric tube and its insertion method. For example, the nasal anchor 80 may be inserted into patient's nostril to form a protected path inside patient's nasal cavity 10 (area around line A-A). Next, the nasogastric tube 50 may be inserted through the nasal anchor 80, pass the oropharynx area 14 (area around line B-B), through the gullet (esophagus) 16, and further toward stomach 18 of the patient. In some embodiments, a distal balloon 133 improves floating of the nasogastric tube in the stomach fluid. Numeral 52 signifies a proximal end of the nasogastric tube 50 (viewed from the operator's point of view), and numeral 54 signifies a distal end of the nasogastric tube.

Due to the presence of the nasal anchor 80, patient's nasal cavity remains protected against rubbing, scratching and/or other irritation during the insertion of the nasogastric tube 50. However, in some cases the nasal cavity 10 remains sensitive to both the nasal anchor 80 and the nasogastric tube 50, because, for example, the nasal anchor and nasogastric tube are bent in that area, therefore causing pressure and irritation of the tissue. Furthermore, rubbing and/or bending of the nasogastric tube 50 through the oropharynx area 14 may cause additional irritation and pain of the tissue. In addition to remedying irritation and pain, different medical procedures may require delivery of specific medications to the nasal cavity 10 and/or oropharynx area 14. Therefore, aside from a basic function of the nasogastric tube 50 (e.g., evacuation of the stomach fluid), the inventive nasal anchor 80 and nasogastric tube 50 are also suitable for delivering therapeutic agents (anesthetics, medications, etc.) into different zones along the path of the nasogastric tube 50. Nasal cavity 10 may be referred to as a nasal treatment area, intranasal treatment area, intranasal mucosa area or a first treatment area. Oropharynx area 14 may be referred to as a second treatment area.

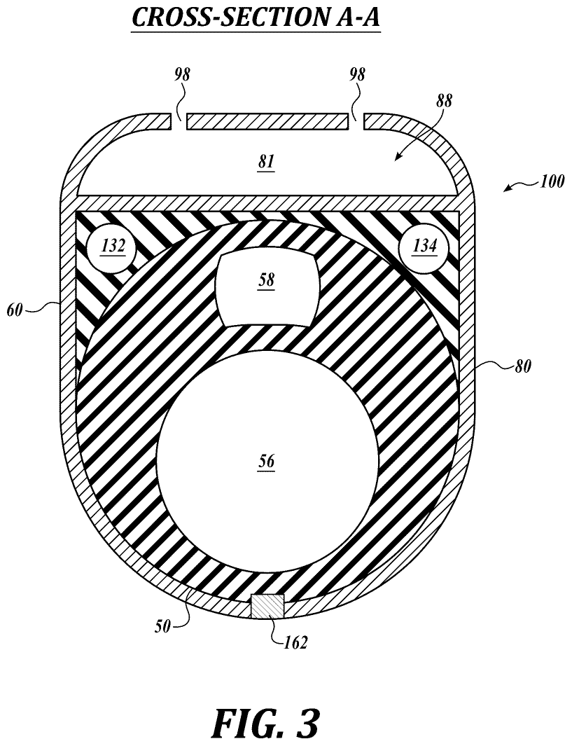

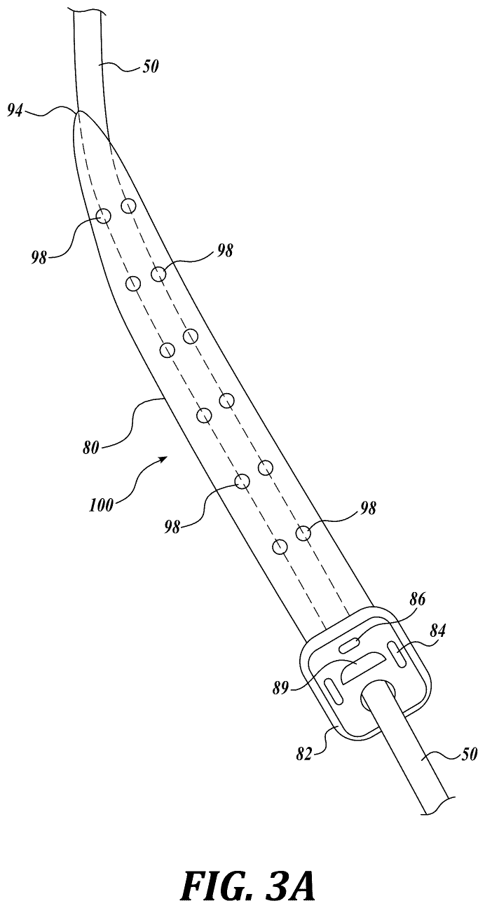

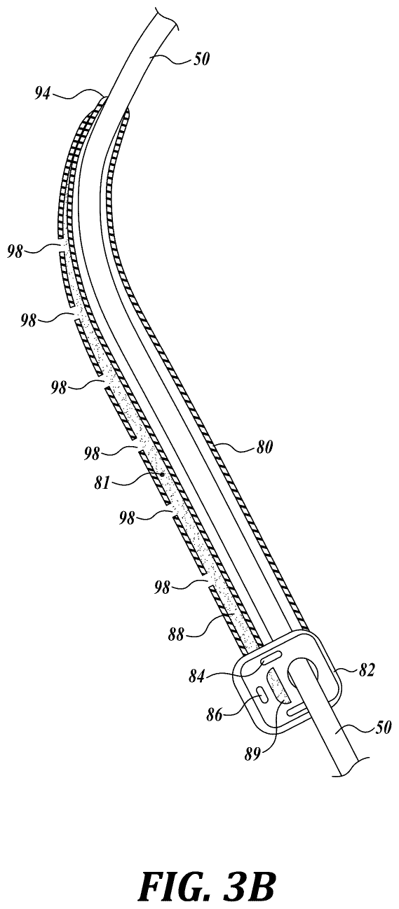

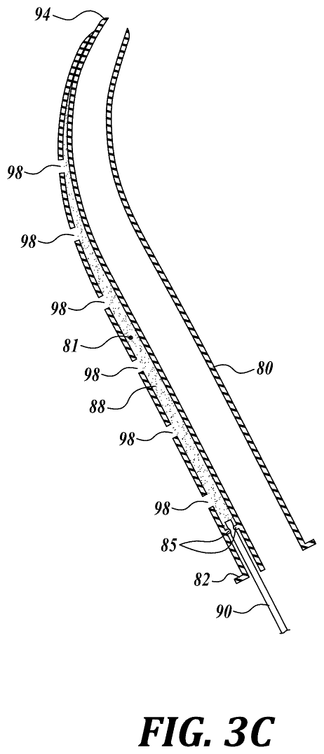

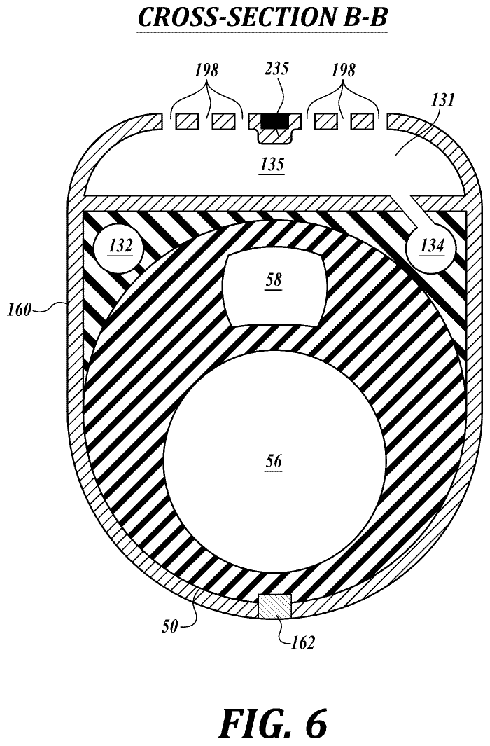

FIG. 3 is a cross-sectional view A-A of a combination of nasogastric tube and nasal anchor shown in FIG. 2. When the nasogastric tube assembly 100 is in its operational position, the cross-sectional plane A-A is located within the nasal cavity 10. In some embodiments, a lumen 56 is an evacuation lumen configured for evacuating stomach fluid, and a lumen 58 is a gas lumen configured for adding gas to the stomach of the patient in order to manage the negative pressure caused by the evacuation of the stomach fluid. Lumen 132 may be used for pressurizing the distal balloon inside the patient's stomach. Lumen 134 may be used for delivering therapeutic agent to another treatment area, for example to the oropharynx treatment area, as further explained in conjunction with FIG. 6 below.

A person of ordinary skill would know that the nasal anchor and nasogastric tube may be made of several suitable materials, for example, silicon, polyurethane, polyvinylchloride (PVC), or other materials. In some embodiments, the nasogastric tube includes a visual marker 162 (also referred to as a radio-opaque line) for ease of orientation of the nasogastric tube against the target treatment areas. For example, the visual marker 162 may be configured to guide the operating physician in orienting the visual marker toward the chin of the patient. In turn, such orientation of the visual marker 162 assures a proper orientation of releasing the therapeutic agent against the tissue of the target area.

As explained above, the nasal cavity 10 may be prone to tissue irritation or trauma. In the embodiment of inventive technology, when the nasogastric tube 50 is inserted through the nasal anchor 80, the passage of the nasogastric tube does not irritate the tissue protected by the nasal anchor. Furthermore, in some embodiments, the nasal anchor may be softer than the nasogastric tube, thus the patient's tissue contacts a relatively soft surface 60 of the nasal anchor 80, as opposed to a relatively hard surface of the nasogastric tube 50, therefore additionally helping to relieve irritation of the tissue. However, even though the nasal anchor 80 protects the nasal cavity against rubbing and scratching by the nasogastric tube, the pressure imparted on the nasal cavity by the nasogastric tube 50 and/or nasal anchor 80 may still cause discomfort to the patient. Therefore, in some embodiments of the present technology, the nasal anchor 80 includes a first space (also referred to as a pocket) 88 that in operation receives a first therapeutic agent (e.g., an anesthetic) 81, which is releasable toward the first treatment area (e.g., nasal cavity) through the openings (apertures) 98.

In different embodiments, the therapeutic agents may include anesthetics, topical local anesthetics, chemotherapy drugs, or other drugs. In some embodiments, such therapeutic agents may be administered in a gelatinous formulation configured for improved adhesion to patient's tissue in the first target area and/or the second target area.

FIG. 3A is a side view of an assembly 100 of nasal anchor 80 and nasogastric tube 50 in accordance with an embodiment of the present technology. The nasal anchor may include a flange 82 at its proximal end to limit the insertion depth, and a curved end 94 at its distal end to limit scratching of the tissue during insertion. The nasal anchor 80 includes the openings 98 (e.g., holes, slits, etc.) for releasing the first therapeutic agent toward the target tissue. Two parallel rows of the openings 98 are illustrated, however, a person of ordinary skill would understand that other arrangement of openings are also encompassed by the inventive technology. In some embodiments, the two rows of the first apertures may be located at approximately 1 o'clock position and 11 o'clock position, thereby assuring delivery of the first therapeutic agent at the first treatment area even in case of some amount of rotational positioning error of the nasal anchor 80. In different embodiments, the nasal anchor may include different numbers of rows of the first apertures (e.g., 1, 3, 4 or other numbers).

FIG. 3B is a cross-sectional side view of a combination of nasal anchor and nasogastric tube in accordance with embodiments of the present technology. In some embodiments, first therapeutic agent 81 enters the first space (pocket) 88 through the inlet opening 89, followed by releasing the first therapeutic agent 81 through a plurality of openings 98. As explained above, proper positioning of the openings 98 may be improved by the operator observing locations of the visual marker 162 at the anterior side of the nasogastric tube 50 and/or flattened posterior area of the nasal anchor.

FIG. 3C is a cross-sectional side view of therapeutic agent delivery in accordance with embodiments of the present technology. In some embodiments, the first therapeutic agent 81 may be delivered into the first space 88 through a medication straw 90. When inserted into the first space 88, the medication straw 90 presses against internal flaps 85 of the nasal anchor, therefore preventing or at least limiting leakage of the first therapeutic agent out of the first space 88. Thus, the openings 98 remain the only available outlet for the first therapeutic agent 81 out of the first space 88 toward the target tissue.

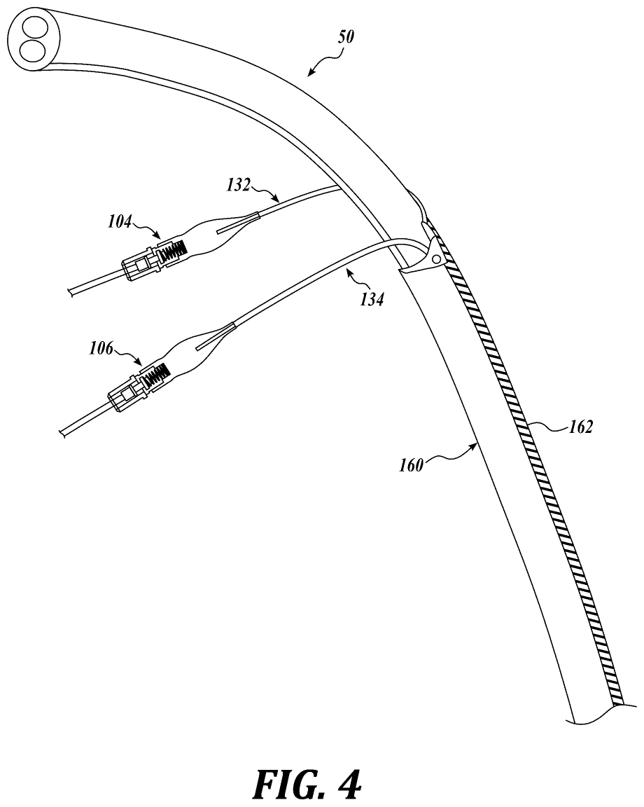

FIG. 4 is a partial side view of a nasogastric tube in accordance with an embodiment of the present technology. In some embodiments, the nasogastric tube 50 may be wrapped in a thin layer of a pliable material 160 to minimize irritation of the surrounding tissue and to facilitate application of a second therapeutic agent, as described in more detail below. In operation, when the nasogastric tube 50 is positioned in its target location, a visual marker 162 on the pliable material (also referred to as soft wrapping) 160 may indicate the posterior side of the assembly. The visual marker 162 helps alignment of the spaces (pockets) for the therapeutic agent deliver against the target areas, as explained in more detail below.

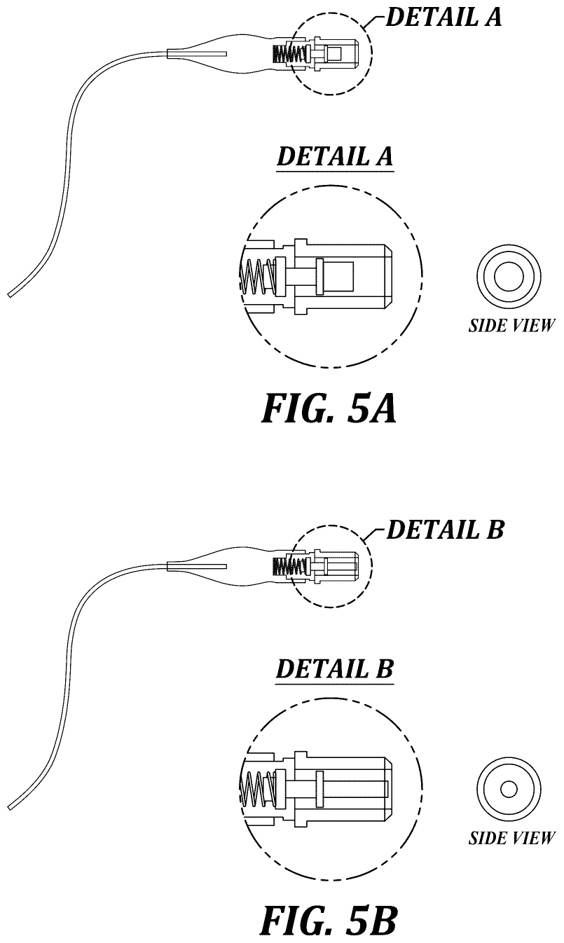

In some embodiments, the nasogastric tube assembly includes Luer locks 104 and 106 that are respectively connected to a distal balloon lumen 132 and a second treatment lumen 134. In practical applications, the illustrated lumens 132 and 134 are generally longer than illustrated in this drawing, the shortened lumens being shown to simplify and clarify the drawings. The Luer locks 104, 106 are just one embodiment of suitable mechanism for delivering therapeutic agents, delivering gas for distal or other balloons, and other fluid delivery or evacuation. In other embodiments, different mechanisms that are used for fluid delivery or evacuation are still within the scope of the inventive technology. Details about Luer locks are illustrated in FIGS. 5A and 5B below.

FIGS. 5A and 5B are detail views of Luer locks in accordance with embodiments of the present technology. Illustrated Luer locks control one or more fluids administered into or out of patient's body through suitable lumens. In some embodiments, Luer locks include check valves that enable safe disconnection from the lumens without spillage or loss of the fluids, or at least with minimized spillage or loss.

FIG. 6 is a cross-sectional view B-B of a nasogastric tube shown in FIG. 2. When the nasogastric tube assembly 100 is in its operational position, the cross-sectional plane B-B is located within the oropharynx area, which is another possible treatment area for applying a therapeutic agent. The oropharynx area 14 may be referred to as an oropharynx treatment area, an oropharynx mucosa area or a second treatment area. In some embodiments, the second therapeutic agent 131 is transferred through a second treatment lumen 134 into a second space (pocket) 135. In some embodiments, the second space 135 is a distensible balloon that expands and receives the second therapeutic agent through the Luer lock 106 and the second treatment lumen 134. As the pressure of second therapeutic agent increases within the second space 135, openings 198 (e.g., micro-apertures) also increase and start releasing the second therapeutic agent toward the oropharynx area. When the second pocket 135 is not pressurized by adding the second therapeutic agent into the pocket, the second pocket is in its deflated position, therefore requiring relatively little additional space to the cross-section of the nasogastric pipe itself. As a result, the combination of the nasogastric tube 50 and the pliable material 160 with the pocket 135 in its deflated position can relatively easily pass through the nasogastric anchor.

In some embodiments, the pliable material 160 includes a strengthening element 235 (also referred to as a strength band) that increases rigidity of the device, therefore improving handling and positioning of the nasogastric tube. In some embodiments, the strengthening element 235 is made of high strength polyurethane or other material that is more rigid than the material of the nasal anchor itself. A rotational positioning of the nasogastric tube with respect to the target tissue of the patient may be further improved by the visual marker 162 at the anterior side of the nasogastric tube.

In different embodiments, the second therapeutic agents may include anesthetics, topical local anesthetics, chemotherapy drugs, or other drugs. In different embodiments, the first and second therapeutic agents may be same or different.

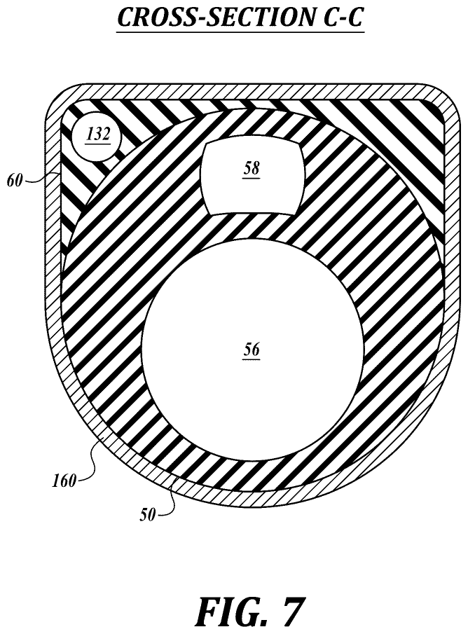

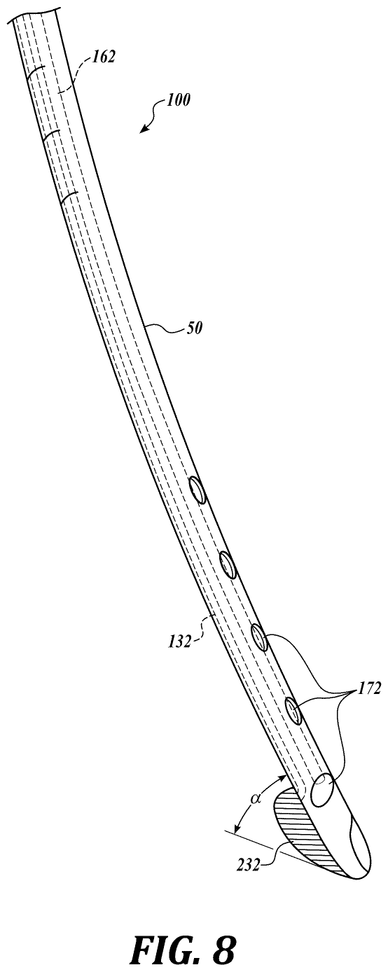

FIG. 7 is a cross-sectional view C-C of a nasogastric tube shown in FIG. 2. As explained above, stomach fluid is evacuated through the lumen 56, and the stomach gas is provided through the lumen 58. Lumen 132 may be used for inflating the distal balloon, as explained below with respect to FIG. 8.

FIG. 8 is a side view of nasogastric tube in accordance with an embodiment of the present technology. In some embodiments, a distal balloon 232 is configured to lift the openings 172 for the suction and sump venting functions away from the gastric mucosa. In some embodiments, the distal balloon 232 may be wedged as shown by angle α to improve the floatation effect. The distal balloon 232 may be inflated through the distal balloon lumen 132.

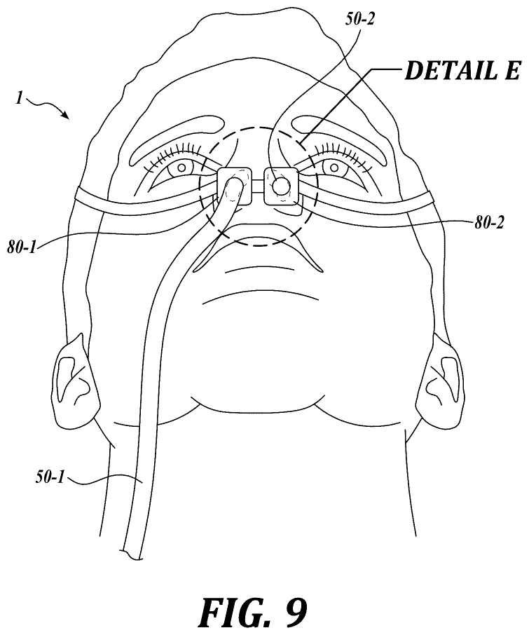

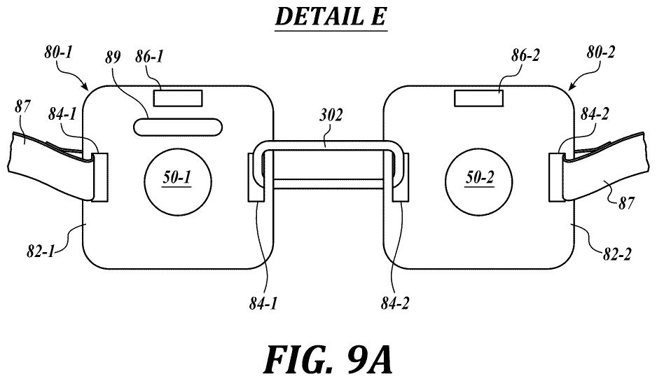

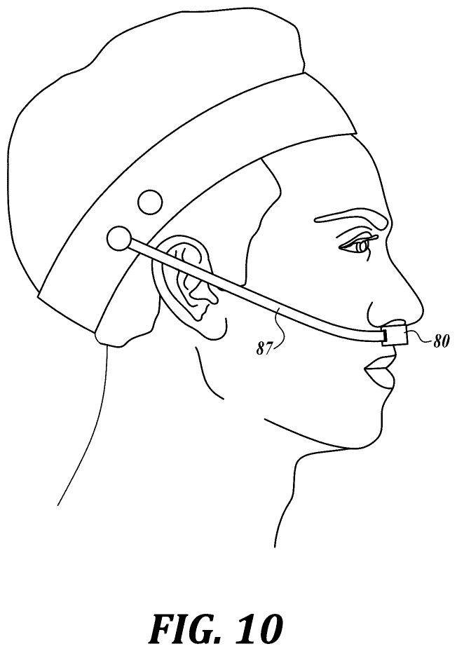

FIG. 9 is an isometric view of an attachment of a nasal anchor and nasogastric tube in accordance with an embodiment of the present technology. Two nasal anchors are used with the illustrated patient. The numerals in FIGS. 9, 9A and 10 are assigned such that the ‘-1’ numerals correspond to one nasal anchor, and the ‘-2’ numerals correspond to another nasal anchor.

In some embodiments, one nasal anchor 80-1 may be used for guiding a nasogastric tube 50-1, whereas the other nasal anchor 80-2 is used for providing oxygen to the patient. Providing oxygen to the patient may help to reduce a feeling of dyspnea that often occurs when one side of the nose is obstructed, as happens when a nasogastric tube is in its place. For the purposes of the oxygen delivery, the nasal anchor 80-2 extends into the nostril of the patient to generally shorter distance than the nasal anchor that guides the nasogastric tube. For example, the nasal anchor 80-2 may extend about 1 cm into the nostril of the patient. Such an arrangement of nasal anchors and their respective roles may be beneficial both for improved comfort of the patient and also for improved attachment of the device to the patient, as described below with respect to FIG. 9A.

FIG. 9A is a detail view of an attachment of a nasal anchor in accordance with an embodiment of the present technology. With the illustrated embodiment, nasal anchor 80-1 guides the nasogastric tube, and nasal anchor 80-2 guides an oxygen canula (not shown). In operation, the nasal anchors 80-1 and 80-2 may be kept at their respective locations by a nasal clip 302 that passes through to the pairs of slits 84 and/or 86. In some embodiments, the nasal clip 302 provides an improved retention of the nasal anchors in comparison to the conventional method of taping the nasal anchor directly to the skin of the user, which may be susceptible to detachment due to, for example, sweating of the patient. The nasal anchors 80-1 and 80-2 may be kept in place through mooring ropes 87, as described below with respect to FIG. 10.

FIG. 10 is a detail view of an attachment of a nasal anchor in accordance with an embodiment of the present technology. In some embodiments, the mooring ropes 87 pass through their respective lateral slits 84 and then backwards over the ears or over buttons on a headband for better comfort of the patient and better retention of the nasal anchors.

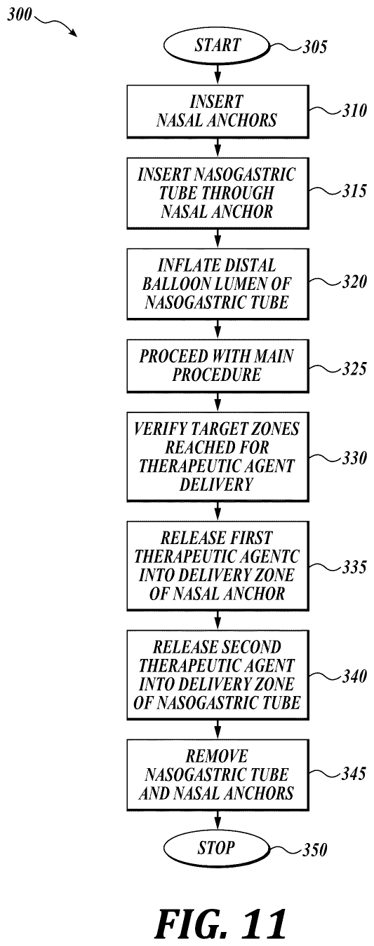

FIG. 11 is a flowchart of a method for inserting and using a nasogastric tube in accordance with an embodiment of the present technology. In some embodiments, the method may include additional steps or may be practiced without all steps illustrated in the flow chart. Furthermore, in different embodiments, the steps of the method may be executed in the order different from that illustrated in FIG. 11.

The method starts in block 305. In block 310, the nasal anchors are inserted into patient's nose. As explained above, one nasal anchor may reduce irritation caused by the insertion of the nasogastric tube, while still enabling delivery of the therapeutic agent (e.g., an anesthetic) to the target treatment area (e.g., nasal treatment area). The other nasal anchor may facilitate delivery of oxygen to the patient. In some embodiment, only one nasal anchor may be used, that is the nasal anchor for the insertion of the nasogastric tube.

In block 315, the nasogastric tube is inserted through the nasal anchor and patent's esophagus, ending in the patient's stomach. Proper orientation of the nasogastric tube may be assured by observing the location of the visual marker (e.g., a radio-opaque line) extending along a body of the nasogastric tube. For example, the visual marker may be configured to point to patient's chain, thus assuring proper orientation of the openings for delivering of the second therapeutic agent.

In block 320, a distal balloon of the nasogastric tube is inflated. In some embodiments, inflation of the distal balloon improves suction and sump venting functions away from gastric mucosa while the nasogastric tube is inserted into the patient. The distal balloon may be inflated through the Luer lock.

In block 325, the main procedure is executed. A non-limiting example of such main procedure is evacuation of the stomach fluid accompanied by adding stomach gas to patient's stomach.

In block 330, an operator verifies that the target zones are properly reached before delivering therapeutic agents (e.g., the first and second therapeutic agents). As a non-limiting example, the first target zone may be the nasal treatment area and the second target zone may be the oropharynx treatment area of the patient. In different embodiments, different numbers and locations of the target zones may be used.

In block 335, the first therapeutic agent is released. As explained with respect to FIGS. 4-4E above, the therapeutic agent may be released through the openings in the nasal anchor.

In block 340, the second therapeutic agent is released toward the second treatment area. Some non-limiting examples of the first and second therapeutic agents are anesthetics, topical local anesthetics, and chemotherapy drugs.

In block 345, the nasogastric tube and nasal anchor are removed. The method ends in block 350.

Although the present invention is defined in the claims, it should be understood that the present invention can alternatively be defined in accordance with the following examples:

Example 1. A device for administering therapeutic agent to a patient's target area, the device comprising:
a nasal anchor configured for insertion into a nose of the patient, the nasal anchor comprising a first pocket in fluid communication with at least one first opening configured for delivering a first therapeutic agent to a first target area of the patient;
a nasogastric tube configured for insertion through the nasal anchor into a stomach of the patient; and
a wrapping disposed about the nasogastric tube, wherein the wrapping comprises a second pocket in fluid communication with at least one second opening configured for delivering a second therapeutic agent to a second target area of the patient.

Example 2. The device of example 1, further comprising a medication straw fluidly connected to the nasal anchor, wherein the medication straw is configured for delivering the first therapeutic agent to the first pocket.

Example 3. The device of example 2, wherein the medication straw is detachable, and wherein the medication straw is fluidly sealed against internal flaps of the nasal anchor.

Example 4. The device of example 1, wherein the at least one first opening of the first pocket comprises multiple first openings arranged in two rows.

Example 5. The device of example 4, wherein the two rows of the multiple first openings of the first pocket are arranged approximately along an 11 o'clock position and a 1 o'clock position.

Example 6. The device of example 1, further comprising a second treatment lumen fluidly connected to the second pocket, wherein the second treatment lumen is configured for delivering the second therapeutic agent to the second pocket.

Example 7. The device of example 6, further comprising a Luer lock configured to open and close delivery of the second therapeutic agent to the second pocket.

Example 8. The device of example 1, further comprising:
an evacuation lumen extending substantially through an entire length of the nasogastric tube, wherein the evacuation lumen is configured for evacuating bodily fluids from the stomach of the patient; and
a stomach gas lumen configured to provide gas to enter the stomach of the patient in response to a negative pressure in the stomach.

Example 9. The device of example 8, wherein the nasogastric tube further comprises:
a distal balloon configured for lifting openings for suction and sump venting functions away from gastric mucosa while the nasogastric tube is inserted into the patient; and
a distal balloon lumen configured for inflating the distal balloon.

Example 10. The device of example 1, wherein the first target area is an intranasal mucosa area of the patient and the second target area is an oropharyngeal mucosa area of the patient.

Example 11. The device of example 1, wherein the first therapeutic agent and the second therapeutic agent are selected from a group consisting of anesthetics, topical local anesthetics, and chemotherapy drugs.

Example 12. The device of example 1, wherein the first therapeutic agent and the second therapeutic agent are supplied in a gelatinous formulation configured for adhesion to patient's tissue in the first target area and the second target area, respectively.

Example 13. The device of example 1, wherein the first therapeutic agent and the second therapeutic agent are same.

Example 14. The device of example 1, further comprising a visual marker extending along the nasogastric tube, wherein, in an operational position, the visual marker faces anteriorly towards a chin of the patient.

Example 15. The device of example 1, further comprising a strengthening element extending along the wrapping, wherein, in an operational position, the strengthening element faces posteriorly away a chin of the patient.

Example 16. A method for administering at least one therapeutic agent to a patient's target area, the method comprising:
inserting a nasal anchor into a nose of the patient:
inserting a nasogastric tube through the nasal anchor into a stomach of the patient,
delivering a first therapeutic agent to a first pocket disposed on the nasal anchor;
delivering the first therapeutic agent to a first target area of the patient through at least one first opening of the first pocket; and
delivering a second therapeutic agent to a second pocket of a wrapping of the nasogastric tube;
delivering the second therapeutic agent to a second target area of the patient through at least one second opening of the wrapping.

Example 17. The method of example 16, wherein the nasal anchor is a first nasal anchor inserted into a first nostril of the patient, the method further comprising:
inserting a second nasal anchor into a second nostril of the patient; and
delivering oxygen to the patient through the second nasal anchor.

Example 18. The method of example 17, further comprising:
connecting the first nasal anchor and the second nasal anchor with a clip.

Example 19. The method of example 18, further comprising:
securing the first nasal anchor and the second nasal anchor to patient's head with mooring ropes attached a headband.

Example 20. The method of example 16, further comprising:
inserting a medication straw into the first pocket, wherein the medication straw is configured for delivering the first therapeutic agent to the first pocket, and wherein the medication straw is fluidly sealed against internal flaps of the nasal anchor.

Example 21. The method of example 16, wherein the at least one first opening of the first pocket comprises multiple first openings arranged in two rows.

Example 22. The method of example 21, wherein the two rows of the multiple first openings of the first pocket are arranged approximately along an 11 o'clock position and a 1 o'clock position.

Example 23. The method of example 16, further comprising delivering the second therapeutic agent to the second pocket through a second treatment lumen.

Example 24. The method of example 23, further comprising opening and closing delivery of the second therapeutic agent to the second pocket further through a Luer lock.

Example 25. The method of example 16, further comprising aligning a visual marker of the nasogastric tube to face anteriorly toward a chin of the patient.

Example 26. The method of example 16, further comprising:
evacuating bodily fluids from the stomach of the patient through an evacuation lumen extending substantially through an entire length of the nasogastric tube; and
in response to a negative pressure in the stomach, providing a stomach gas to the stomach of the patient through a stomach gas lumen.

Example 27. The method of example 16, wherein the first target area is an intranasal mucosa area of the patient and the second target area is an oropharyngeal mucosa area of the patient, and wherein the first therapeutic agent and the second first therapeutic agent are selected from a group consisting of anesthetics, topical local anesthetics, and chemotherapy drugs.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” etc., mean plus or minus 5% of the stated value.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

What is claimed is:

1. A device for administering therapeutic agent to a patient's target area, the device comprising:
a nasal anchor configured for insertion into a nose of the patient, the nasal anchor comprising a first pocket in fluid communication with at least one first opening configured for delivering a first therapeutic agent to a first target area of the patient;
a nasogastric tube configured for insertion through the nasal anchor into a stomach of the patient; and
a wrapping disposed about the nasogastric tube, wherein the wrapping comprises a second pocket in fluid communication with at least one second opening configured for delivering a second therapeutic agent to a second target area of the patient.

2. The device of claim 1, further comprising a medication straw fluidly connected to the nasal anchor, wherein the medication straw is configured for delivering the first therapeutic agent to the first pocket.

3. The device of claim 2, wherein the medication straw is detachable, and wherein the medication straw is fluidly sealed against internal flaps of the nasal anchor.

4. The device of claim 1, wherein the at least one first opening of the first pocket comprises multiple first openings arranged in two rows.

5. The device of claim 4, wherein the two rows of the multiple first openings of the first pocket are arranged approximately along an 11 o'clock position and a 1 o'clock position.

6. The device of claim 1, further comprising a second treatment lumen fluidly connected to the second pocket, wherein the second treatment lumen is configured for delivering the second therapeutic agent to the second pocket.

7. The device of claim 6, further comprising a Luer lock configured to open and close delivery of the second therapeutic agent to the second pocket.

8. The device of claim 1, further comprising:
an evacuation lumen extending substantially through an entire length of the nasogastric tube, wherein the evacuation lumen is configured for evacuating bodily fluids from the stomach of the patient; and
a stomach gas lumen configured to provide gas to enter the stomach of the patient in response to a negative pressure in the stomach.

9. The device of claim 8, wherein the nasogastric tube further comprises:
a distal balloon configured for lifting openings for suction and sump venting functions away from gastric mucosa while the nasogastric tube is inserted into the patient; and
a distal balloon lumen configured for inflating the distal balloon.

10. The device of claim 1, wherein the first target area is an intranasal mucosa area of the patient and the second target area is an oropharyngeal mucosa area of the patient.

11. The device of claim 1, wherein the first therapeutic agent and the second therapeutic agent are selected from a group consisting of anesthetics, topical local anesthetics, and chemotherapy drugs.

12. The device of claim 1, wherein the first therapeutic agent and the second therapeutic agent are supplied in a gelatinous formulation configured for adhesion to patient's tissue in the first target area and the second target area, respectively.

13. The device of claim 1, wherein the first therapeutic agent and the second therapeutic agent are same.

14. The device of claim 1, further comprising a visual marker extending along the nasogastric tube, wherein, in an operational position, the visual marker faces anteriorly towards a chin of the patient.

15. The device of claim 1, further comprising a strengthening element extending along the wrapping, wherein, in an operational position, the strengthening element faces posteriorly away a chin of the patient.

16. A method for administering at least one therapeutic agent to a patient's target area, the method comprising:
inserting a nasal anchor into a nose of the patient:
inserting a nasogastric tube through the nasal anchor into a stomach of the patient,
delivering a first therapeutic agent to a first pocket disposed on the nasal anchor;
delivering the first therapeutic agent to a first target area of the patient through at least one first opening of the first pocket; and
delivering a second therapeutic agent to a second pocket of a wrapping of the nasogastric tube;
delivering the second therapeutic agent to a second target area of the patient through at least one second opening of the wrapping.

17. The method of claim 16, wherein the nasal anchor is a first nasal anchor inserted into a first nostril of the patient, the method further comprising:
inserting a second nasal anchor into a second nostril of the patient; and
delivering oxygen to the patient through the second nasal anchor.

18. The method of claim 17, further comprising:
connecting the first nasal anchor and the second nasal anchor with a clip.

19. The method of claim 18, further comprising:
securing the first nasal anchor and the second nasal anchor to patient's head with mooring ropes attached a headband.

20. The method of claim 16, further comprising:
inserting a medication straw into the first pocket, wherein the medication straw is configured for delivering the first therapeutic agent to the first pocket, and wherein the medication straw is fluidly sealed against internal flaps of the nasal anchor.

21. The method of claim 16, wherein the at least one first opening of the first pocket comprises multiple first openings arranged in two rows.

22. The method of claim 21, wherein the two rows of the multiple first openings of the first pocket are arranged approximately along an 11 o'clock position and a 1 o'clock position.

23. The method of claim 16, further comprising delivering the second therapeutic agent to the second pocket through a second treatment lumen.

24. The method of claim 23, further comprising opening and closing delivery of the second therapeutic agent to the second pocket further through a Luer lock.

25. The method of claim 16, further comprising aligning a visual marker of the nasogastric tube to face anteriorly toward a chin of the patient.

26. The method of claim 16, further comprising:
evacuating bodily fluids from the stomach of the patient through an evacuation lumen extending substantially through an entire length of the nasogastric tube; and
in response to a negative pressure in the stomach, providing a stomach gas to the stomach of the patient through a stomach gas lumen.

27. The method of claim 16, wherein the first target area is an intranasal mucosa area of the patient and the second target area is an oropharyngeal mucosa area of the patient, and wherein the first therapeutic agent and the second first therapeutic agent are selected from a group consisting of anesthetics, topical local anesthetics, and chemotherapy drugs.
